(19) United States
(12) Patent Application Publication
(10) Pub. No.: US 2020/0175674 A1
(43) Pub. Date: Jun. 4, 2020

(54) QUANTIFIED ASPECTS OF LESIONS IN MEDICAL IMAGES

(71) Applicant: KONINKLIJKE PHILIPS N.V., EINDHOVEN (NL)

(72) Inventors: RAFAEL WIEMKER, KISDORF (DE); HEIKE RUPPERTSHOFEN, AHRENSBURG (DE); NICOLE SCHADEWALDT, NORDERSTEDT (DE); TOBIAS KLINDER, UELZEN (DE)

(21) Appl. No.: 16/616,125

(22) PCT Filed: May 25, 2018
(86) PCT No.: PCT/EP2018/063806

§ 371 Date: Nov. 22, 2019

Related U.S. Application Data
(60) Provisional application No. 62/514,046, filed on Jun. 2, 2017.

(51) Int. Cl. G06T 7/00; G06T 7/11; G06T 7/73; G16H 30/40

(52) U.S. Cl. CPC G06T 7/0012; G06T 7/11; G06T 7/74; G06T 2207/10088; G06T 2207/30096; G06T 2207/10081; G16H 30/40

(57) ABSTRACT

A system (100) comprises a segmenter (130) and a quantification tool (140). The segmenter segments a lesion (102) in a medical image (104). The quantification tool (140) quantifies an aspect of the segmented lesion according to a set of parameters, wherein the quantified aspect includes spiculation, heterogeneity, vascularization or combinations thereof.

### FIELD OF THE INVENTION

The following generally relates to quantifiable aspects of abnormal growths or lesions in medical images, such as computed tomography (CT), magnetic resonance (MR), Ultrasound (US), and the like, and more specifically to quantifying imaging aspects of lesions, such as spiculation, hetereogeneity and/or vascularization.

### BACKGROUND OF THE INVENTION

Lesions, such as lung nodules or tumors, are a substance in a body, whose presence in a medical image is indicative of a disease, such as cancer. Conventionally, aspects of the lesions can be qualitatively described, such as spiculated and/or vascularized.

Qualitative aspects of lesions are often described with relative degrees, such as highly or poorly, very and little, and the like. Spiculated lesions are tissues with spikes or points on a surface, which are suggestive but not diagnostic of malignancy.

A highly-vascularized lesion or poorly vascularized lesion refer to degrees of blood supply to a tumor. The blood can supply nutrients to a tumor, which is a consideration for tumor growth and/or transport, such as metastasis.

Heterogeneity or non-uniformity in tissues of a tumor, such as density, are noted in many types of cancer. Conventional practice is to measure tumor heterogeneity by a computed entropy, which is a singular measure of disorder of the tissue.

### SUMMARY OF THE INVENTION

Aspects described herein address the above-referenced problems and others.

The following describes embodiments of a system and method for quantifying aspects of lesions present in medical images, such as spiculation, vascularization, heterogeneity, and combinations thereof The quantified aspects use computed measurements of segmented lesions with a result that can include an individual score or with greater granularity, feature vectors. The computed measurements include parameters, which can be varied to identify, with precision, characteristic features in medical images of lesions. In some instances, the flexibility of parameters and computational efficiency provide effective research tools that use the quantified aspects to determine features of diagnostic or predictive value.

In one aspect, a system comprises a segmenter and a quantification tool. The segmenter segments a lesion in a medical image. The quantification tool quantifies an aspect of the segmented lesion according to a set of parameters, wherein the quantified aspect includes spiculation, heterogeneity, vascularization or combinations thereof.

In another aspect, a method comprises segmenting a lesion in a medical image. An aspect of the segmented lesion is quantified according to a set of parameters, wherein the quantified aspect includes spiculation, heterogeneity, vascularization, and combinations thereof.

In another aspect, a non-transitory computer-readable storage medium carrying instructions controls one or more processors to segment a lesion in a medical image, and quantify an aspect of the segmented lesion according to a set of parameters, wherein the quantified aspect includes spiculation, heterogeneity, vascularization, and combinations thereof.

These and other aspects of the invention will be apparent from and elucidated with reference to the embodiment(s) described hereinafter.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of a system for quantifying aspects of a lesion in a medical image.

FIG. 2 illustrates an example of a displayed segmented lesion with patches for quantifying spiculation.

FIG. 3 illustrates an example of a non-spiculated patch score and a spiculated patch score.

FIG. 4 illustrates an example of a graph of a bilateral iterative filtering of two different simulated segmented lesions.

FIG. 5 illustrates an example of graphs of a bilateral iterative filtering of two different simulated segmented lesions.

FIG. 6 illustrates an example segmented lesion contour with an inner and outer dimensioned contour.

FIG. 7 illustrates example histograms of joint entropy for a simulated lesion with three different vascularizations.

FIG. 8 flowcharts an embodiment of a method for quantifying aspects of a lesion in a medical image.

FIG. 9 flowcharts an embodiment of a method for quantifying spiculation of a lesion in a medical image.

FIG. 10 flowcharts an embodiment of a method for quantifying heterogeneity of a lesion in a medical image.

FIG. 11 flowcharts an embodiment of a method for quantifying vascularization of a lesion in a medical image.

### DETAILED DESCRIPTION OF EMBODIMENTS

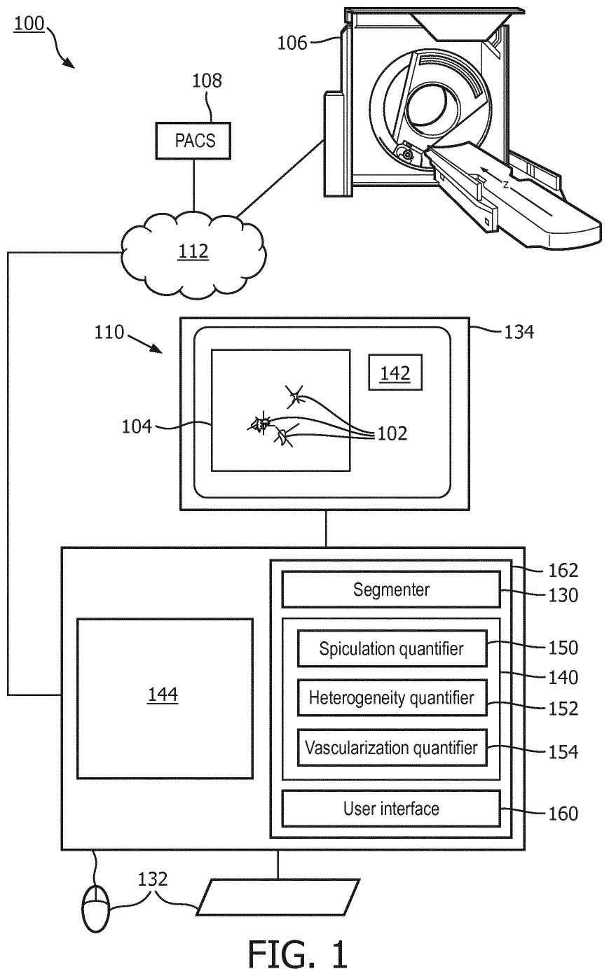

With reference to FIG. 1, an embodiment of a system 100 for quantifying aspects of a lesion 102 in a medical image 104 is schematically illustrated.

The medical image 104 is generated by a medical imaging device 106, such as a CT scanner, a MR scanner, an US device, and the like. The medical image 104 represents a portion of an anatomy of a patient (not shown), which includes one or more lesions 102. The generated medical image 104 can be received directly from the medical imaging device 106 after scanning the patient, or from a storage subsystem 108, such as a Picture Archiving and Communication System (PACS), Radiology Information System (RIS), Electronic Medical Record (EMR), Hospital Information System (HIS), or the like. The medical image 104 can be two dimensional (2D), three dimensional (3D), an image slice, a frame of a video, combinations thereof, and the like.

The medical image 104 is received by a computing device 110, such as a console of the medical imaging device 106, workstation, server, laptop, tablet, body worn computing device, smartphone, distributed computing device, combinations thereof, and the like. The computing device 110 is communicatively connected to the medical imaging device 106 and/or storage subsystem 108 via a network 112, which can be public or private, wired or wireless, data or cellular, combinations thereof, and the like.

A segmenter 130 segments the lesion 102 in the medical image 104. The segmented lesion can be represented by contour line in 2D or a surface in 3D, such as a mesh. The segmenter 130 uses known techniques for segmenting the lesion 102, such as clustering, edge detection, region-growing, graph partitioning, watershed, model based, and the like. The segmenter 130 can be invoked in response to receiving the medical image 130 or manually invoked in response to an input from an input device 132 identifying at least a portion of the lesion 102 in a display of the medical image 104 on a display device 134.

A quantifier tool 140 quantifies an aspect of the segmented lesion 102, which includes a result 142 of a single score or value, and/or a set of feature vectors computed using a set of parameters, Pᵢ where i≥2. The result 142 can be displayed on the display device 134 and/or stored in a non-transitory computer memory 144. In some instances, the result 142 provides for further research over conventional practice of qualitative measures and/or a single score. The quantifier tool 140 includes at least one of a spiculation quantifier 150, a heterogeneity quantifier 152 or a vascularization quantifier 154.

The quantifier tool 140 can be invoked in response to completion of segmentation of the lesion 102 or manually invoked with an input from the input device 132. Likewise, the spiculation quantifier 150, the heterogeneity quantifier 152, and/or the vascularization quantifier 154 can be similarly invoked. The spiculation quantifier 150, the heterogeneity quantifier 152, and/or the vascularization quantifier 154 can be invoked individually or in different combinations. For example, system or user parameters for automatic invocation of each of the spiculation quantifier 150, the heterogeneity quantifier 152, and/or the vascularization quantifier 154. The system or user parameters can include basis in the metadata of the medical image 104, such as anatomy in a DICOM header. For example, with the medical image 104 including lung anatomy in a DICOM header, the spiculation quantifier 150 and the vascularization quantifier 154 are automatically invoked upon completion of segmentation of the lesion 102, while the heterogeneity quantifier 152 is manually invoked or invoked for a lesion 102 greater than a threshold volume or area.

A user interface 160 configures a display and receives input from the input device 132 of the system or user parameters, the parameters of each of the quantifiers 150, 152, 154, display of the medical image 104, the result(s) 142, the input for manual segmentation, the input for invocation of the quantifier tool or individual quantifiers, and the like. The input device 132 is suitably embodied by a keyboard, a mouse, a microphone, and the like. The display device 134 is suitably embodied by a computer display, smartphone display, projector, body worn display, and the like.

The selection of which of the spiculation quantifier 150, the heterogeneity quantifier 152, and/or the vascularization quantifier 154 to invoke and/or which of the corresponding result(s) 142 to display can be based on a protocol, a user preference, a manual selection or combinations thereof. For example, the medical image 104 is received for an imaging protocol that evaluates treatment known tumor for a patient undergoing radiotherapy. The heterogeneity quantifier 152, and the vascularization quantifier 154 are automatically invoked based on a protocol for known tumors to quantify the vascularization and heterogeneity of the known tumor in the medical image 104. In response to spiculation being observed in the medical image 104, the spiculation tool 150 is manually invoked. The results of the heterogeneity quantifier 152, and the vascularization quantifier 154 and displayed according to the protocol. The result of the spiculation tool 150 is displayed according to a user preference that sets a minimum threshold value for the displayed spiculation result.

The computing device 110 or console can be part of, combined with, or separated from the medical imaging scanner 106. The computing device 110 includes one or more configured processors 162, such as a digital processor, a microprocessor, an electronic processor, an optical processor, a multi-processor, a distribution of processors including peer-to-peer, parallel or cooperatively operating processors, client-server arrangement of processors, and the like. The arrangement can include the network 112, which can include a bus structure or other internal or local communication structure.

The computing device 110 includes the processor 162 and the memory 144. The memory 144 is suitably embodied by a configured electronic storage medium, such as local disk, cloud storage, server storage, remote storage and the like, accessed by the configured processor 162. The configured electronic storage medium can include system file structures, relational and/or object oriented database system structures, data structures, and the like.

The segmenter 130, the quantifier tool 140, the spiculation quantifier 150, the heterogeneity quantifier 152, the vascularization quantifier 154, and the user interface 160 are suitably embodied by the processor 162, configured to receive and segment the medical image 104, receive parameter inputs, quantify aspects of the segmented lesion 102, and display and/or store the result 142.

The configured processor 162 executes at least one computer readable instruction stored in the computer readable storage medium 144, such as an optical disk, a magnetic disk, semiconductor memory of a computing device with the configured processor, which excludes transitory medium and includes physical memory and/or other non-transitory medium to perform the disclosed techniques. The configured processor may also execute one or more computer readable instructions carried by a carrier wave, a signal or other transitory medium. The lines between components represented in the diagram represent communications paths.

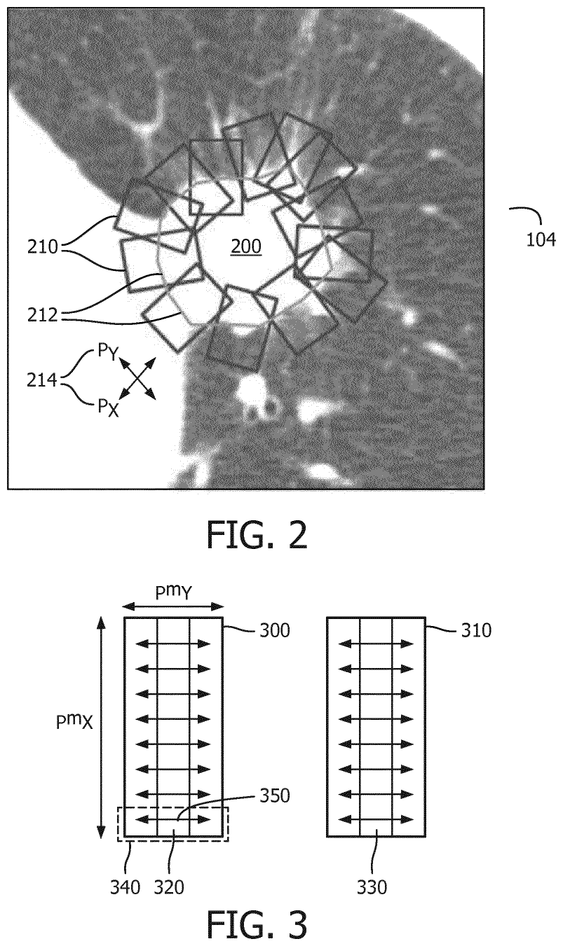

With reference to FIG. 2, an example of a displayed segmented lesion 200, such as a lung nodule, with patches 210 for quantifying spiculation is illustrated. A contour line 212 illustrates a 2D segmentation boundary of the segmented lesion 200.

The patches 210 are rectilinear in shape and include parameterized dimensions, Pₓ and P_{y}, such as Pₓ=3 pixels and P_{y}=5 pixels. The patches 210 are distributed equidistant along the contour line 212 or segmentation boundary with a center line or plane tangential to the contour line 212. The parameterized dimensions 214 of the patches 210 are fixed for each spiculation measurement. That is, spiculation is quantified for a lesion using one set of dimensions, e.g. same for each patch 210. The dimensions 214 can be represented as a number of pixels or voxels, or a distance, such as millimeters (mm).

A spiculation score, S_{1,m}, or result 142 is computed as a number of voxel values greater than a threshold difference, T between the corresponding voxel and a fitted line, L_{1,m} for each line of voxels in the patch, Pₘ parallel to the tangential line. That is, the voxels are sampled for each X line, the line, L_{1,m} is fit to the sampled voxels, and residual values between the predicted value according to the fitted line and the actual values are compared to the threshold value, and those voxels with residual values greater than the threshold are counted for the line spiculation score S_{1,m}, where 1 is the line and m is the patch. The patch score, Sₘ is computed as the sum of the line scores for the patch, and the lesion spiculation score or result is the sum of the spiculation scores of the patches 210.

Thus, the spiculation parameters include the patch dimensions and the threshold. The spiculation result can be represented as a single value or a feature vector. For example, a vector for lesion A in a two dimensional can be represented with a feature vector (A, Pₓ, P_{y}, T, S) and/or finer levels of granularity at the level of each patch M with vector (A, M, Pₓ, P_{y}, T, S) or even finer levels at the level of each line L in a patch with the vector (A, M, Pₓ, P_{y}, T, S). In some embodiments, the fitted line can include a low order polynomial, H, which is included in the vector.

In some instances, the vectors provide data that can precisely and flexibly identify characteristics of spiculated lesions over conventional qualitative indicators, which allows further research into aspects of spiculation and diagnostic indications. The vectors can be stored in the computer memory 144, such as a database. The vectors provide the advantage over conventional practice with the capability to more accurately compare spiculation across patient populations.

With reference to FIG. 3, an example of a non-spiculated patch score 300 and a spiculated patch score 310 for a 3 pixel by 8 pixel patch P^{m}. Shaded pixels 320 indicate a zero or null spiculated score, e.g. less than the threshold T, and non-shaded or white pixels 330 indicate a spiculated score of one. The illustrated patch scores 300, 310 identify the corresponding pixels 340 for each set of pixels scored corresponding to each fitted line 350.

As illustrated in the spiculated patch score 310, a spike presents itself as a sharp deviation from a fitted line with more voxel scores for greater length of the spike, e.g. crossing more fitted lines. In some embodiments, the voxel values are limited or pre-screened to a fixed range, which removes noise due to parenchymal background, rib-bones, calcifications and the like. For example, in CT images of a lung, voxels values outside a range between [−700, 0] Hounsfield Units (HU) for a patch 210 are reset to a corresponding end point of the range, such as −700 to 0.

The non-spiculated patch score 300 includes all shaded voxels because no voxel exceeds the threshold T difference between the corresponding voxel and the corresponding fitted line, and thus, the score is zero. The spiculated patch score 310 includes 8 pixels that exceed the threshold T and are indicated in white for a score of 8.

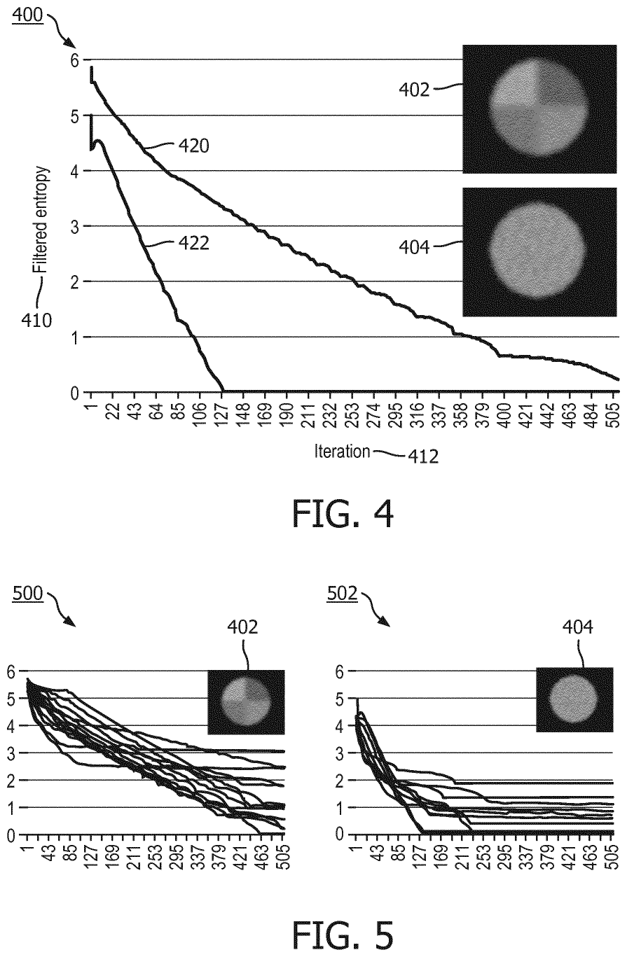

With reference to FIG. 4, an example of a graph 400 of a bilateral iterative filtering of two different simulated segmented lesions 402, 404. The two different simulated segmented lesions 402, 404 each include the same number of pixels with four different gray scale values. The spatial distribution of the four different gray scale values is different, although a Shannon entropy is approximately the same for each of the two different simulated segmented lesions 402, 404. In the first simulated segmented lesion 402, the four different gray scale values are distributed into four separate regions forming slices of a pie shape. In the second simulated segmented lesion 404, the four different gray scale values are uniformly distributed over the simulated lesion.

The heterogeneity quantifier 152 iteratively applies a bilateral filter to the two different simulated segmented lesions 402, 404 and the filtered entropy 410 is computed after each iteration 412. That is, the Shannon entropy is computed using the bilateral filtered voxels in the corresponding simulated segmented lesion 402, 404 for the filtered entropy 410. The bilateral filter, well known in the art, includes a spatial kernel Kₛ which smooths differences in coordinates and a dynamic or range kernel K_{d}, which smooths differences in intensities

A first curve 420 plots the filtered entropies 410 along the vertical axis after each iteration 412 of the bilateral filter using kernels, Kₛ, K_{d} applied to the first simulated segmented lesion 402. A second curve 422 plots the filtered entropies 410 after each iteration 412 of the bilateral filter using same kernels, Kₛ, K_{d} applied to the second simulated segmented lesion 404.

The heterogeneity quantifier 152 computes a heterogeneity score or result 142 for each of the two different simulated segmented lesions 402, 404 as an area under the corresponding curve 420, 422. Thus, the heterogeneity score H of a lesion A can be represented as a feature vector with (A, Kₛ, K_{d}, H), where H is the area under the bilateral smoothing curve iteratively applied using the kernels Kₛ and K_{d}. In some embodiments, the heterogeneity score H can be represented at a finer level of granularity as the entropy value at iteration I. For example, the curves 420, 422 can each be represented with a set of (A, Kₛ, K_{d}, EᵢI) where Eᵢ. is the entropy value at the Ith iteration of the bilateral filtered voxels and I is the Ith iteration of the bilateral filtering of the voxels.

As illustrated with the first curve 420 and the second curve 422, the curves are different and the areas under the corresponding curves are different. The first curve 420 decreases more slowly than the second curve 422. The heterogeneity score either as a single value represented as the area under the curve or as a set of vectors by individual points on the curve illustrate quantitatively the spatial distribution of the gray scale voxels within the corresponding segmented lesion.

With reference to FIG. 5, an example of graphs of a bilateral iterative filtering of the two different simulated segmented lesions 402, 404 is illustrated with different kernels K^{x}ₛ and K^{y}_{d} for each of the two different simulated segmented lesions 402, 404. Each graph includes a vertical axis of the filtered entropy 410 and a horizontal axis of the number of iterations 412. A first graph 500 corresponds to the first segmented lesion 402, and each curve corresponds to a pair (x,y) of the kernels K^{x}ₛ and K^{y}_{d}. For each of the same kernel pairs K^{x}ₛ and K^{y}_{d}, a second graph 502 includes curves using the same pairs (x,y) of the kernels K^{x}ₛ and K^{y}_{d} of the bilateral filter applied to the second segmented lesion 404.

The curves in the first graph 500 differ for the different kernels and also differ between the first graph 500 and the second graph 502 for the same kernel pairs. The variability in the curves within each graph and between graphs illustrates the flexibility of the heterogeneity score to quantify aspects of heterogeneity of a segmented lesion applied at different levels of granularity. The heterogeneity score can be represented with vectors (A, K^{x}ₛ, K^{y}_{d}, H) or even a finer level of granularity with (A, K^{x}ₛ, K^{y}_{d}, Eᵢ, I).

The different vectors used to quantify aspects of segmented lesions 102 have an advantage over other approaches to differentiate quantitatively aspects of different lesions according to selected vectors. For example, research can examine lesions with better diagnostic indications of heterogeneity that correspond to vectors (A, K^{x}ₛ, K^{x}_{d}, Eᵢ, I) which may differ from others of different kernels or entropies of bilateral filtered voxels at different iterations indicative of other less diagnostic lesion tissue density spatial distributions, such as with different spatial and/or density distributions.

In some embodiments, the bilateral filter is expanded to a multilateral filter. The multilateral filter adds n distance kernels K_{g(n)} to the bilateral kernels, where each distance kernel smooths voxels according to distances of voxels from a spectral channel, such as from CT multi-energy or spectral image values. An example vector includes (A, K^{x}ₛ, K^{y}_{d}, K_{g(1)}, . . . K_{g(n)}, H).

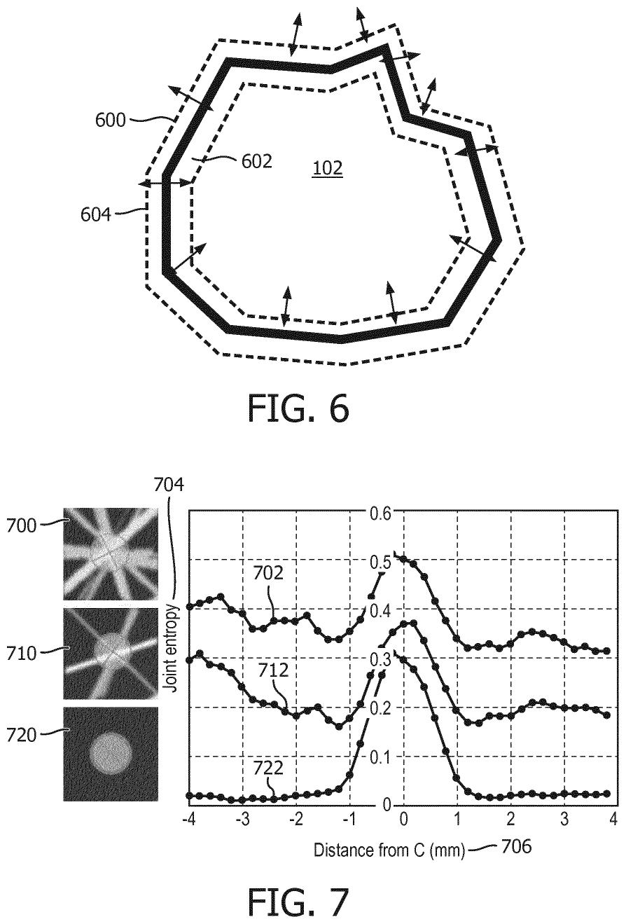

With reference to FIG. 6, an example lesion 102 with a segmented contour 600, an inner dimensioned contour 602, and an outer dimensioned contour 604 is illustrated. The segmented contour 600 C in 2D or segmented surface in 3D is segmented by the segmenter 130. The inner dimensioned contour 602 Cⁱₓ is a contour x distance inside the contour C. The outer dimensioned contour 604 C^{o}_{y} is a contour y distance outside the contour C. In some embodiments, the magnitude of distances x and y are the same. In some embodiments, the magnitudes are different.

The vascularization quantifier 154 samples voxel pairs xᵢ, yᵢi=1 to n which are on a line orthogonal to the segmented contour 600, and xᵢ is a point x distance inside the segmented contour 600 and yᵢ is a point y distance outside the segmented contour 600. The vascularization quantifier 154 computes a joint entropy of the sampled pairs at different distances x,y.

The joint entropy H(X,Y) can be determined from mutual information measures, well known in the art, such as:

$$H ( X , Y ) = {\sum }_{y ∈ Y} {\sum }_{x ∈ X} p ( x , y ) log ( \frac{p ( x , y )}{p ( x ) p ( y )} ) \geq 0 ,$$

where p(x) is a marginal probability distribution of X, p(y) is a marginal probability distribution of Y, and p(x,y) is a joint probability distribution of X and Y. The joint entropy increases according to vessel structures, which continue through the margins of the lesion 102. That is, mutual information measures information about the voxels X and Y with information about one of X and Y reduces uncertainty about the other of X and Y. In some instances, the joint entropy provides quantifiable information across a wide range of lesions independent of vessel size, lesion size, background size, and/or contrast appearances.

With reference to FIG. 7, example histograms of joint entropy for a simulated segmented lesion with three different vascularizations are illustrated. A first image 700 illustrates a simulated highly vascularized segmented lesion and corresponds to a first histogram 702 of joint entropy values 704 according to distances from the segmented contour 706 C. A second image 710 illustrates a simulated medium vascularized segmented lesion and corresponds to a second histogram 712. A third image 720 illustrates a simulated poorly vascularized segmented lesion and corresponds to a third histogram 722. The vertical axis is the joint entropy 704 and the horizontal axis as the distance from C 706.

The vascularization quantifier 154 computes the result 154 for the joint entropy as an area M under the corresponding curve 702, 712, 722. The vector for vascularization can be represented by (A, M), a single value of vascularization M of lesion A, or at a finer granularity with (A, X, Y, J_{x,y}), which includes a set of vectors and J is the joint entropy at distance x, y. In some embodiments, the set of vectors or the area under the joint entropy histogram includes maximum distances for x and/or y.

In some instances, the quantified vascularization provides a measure for research, such as radiomics, that is flexible and can provide comparison across lesions and different patient populations. In some instances, the computational aspects of the computed result 142 include parallelism for efficiency, such as computing the joint entropies at different distances in parallel.

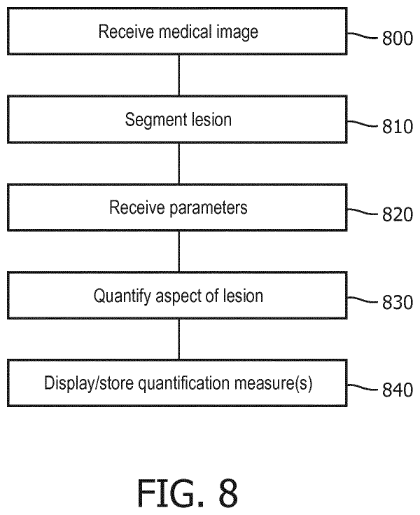

With reference to FIG. 8, an embodiment of a method for quantifying aspects of the lesion 102 in the medical image 104 is flowcharted. At 800, the configured processor 162 receives the medical image 104. The medical image 104 can be received directly from the medical imaging device 106 as generated or from the storage subsystem 108 previously generated by the medical imaging device 106. The medical image 104 can be 2D or 3D, such as a slice, a frame, or a volume.

At 810, the configured processor 162 segments the lesion 102. The segmented lesion includes a boundary, such as a contour line in 2D, a mesh in 3D, and the like. The segmentation can be performed with segmentation algorithms known in the art.

At 820, the configured processor 162 can receive parameters for quantifying aspects of the segmented lesion. The parameters can include the threshold T and the dimensions 214 for patches 210, which are used to quantify spiculation. The parameters can include stopping parameters and kernel parameters, which are used to quantify heterogeneity. The parameters can include inside and outside distances 602, 604, or maximum distances and incremental distances, which are used to quantify vascularization. The parameters can include indicators of a level of granularity or a format of a vector for quantifying spiculation, heterogeneity, vascularization, and combinations thereof.

At 830, the configured processor 162 quantifies using the received parameters at least one aspect of the segmented lesion, such as spiculation, heterogeneity, vascularization, and combinations thereof. The result 142 is returned, which can include a single result or a set of feature vectors. In some embodiments, the configuration of the processor 162 includes the parameters, such as included in instructions of the configured processor 162 and the configured processor 162 quantifies at least one aspect of the segmented lesion.

At 840, the configured processor 162 displays and/or stores the result 142. The result 142 can be displayed on the display device 134. The result 142 can be stored in the computer memory 144, the storage subsystem 108, or other non-transitory computer memory.

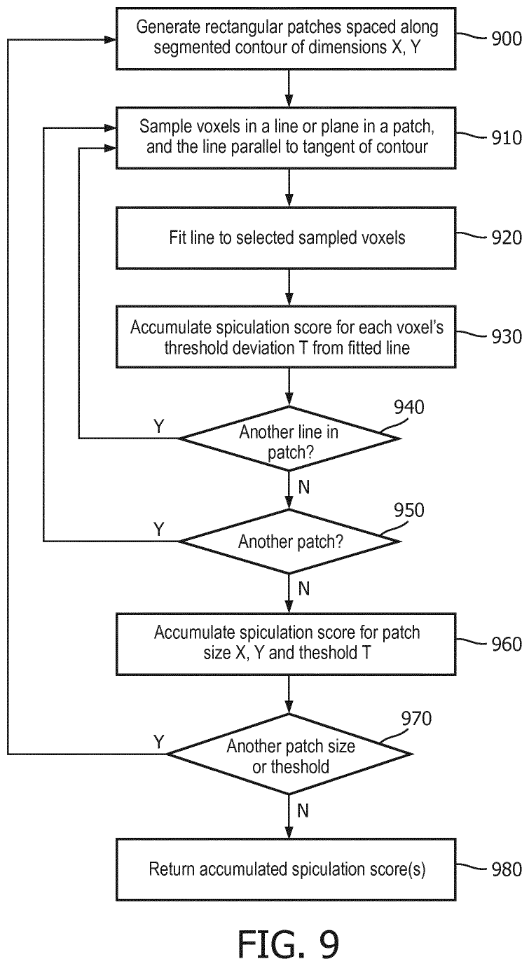

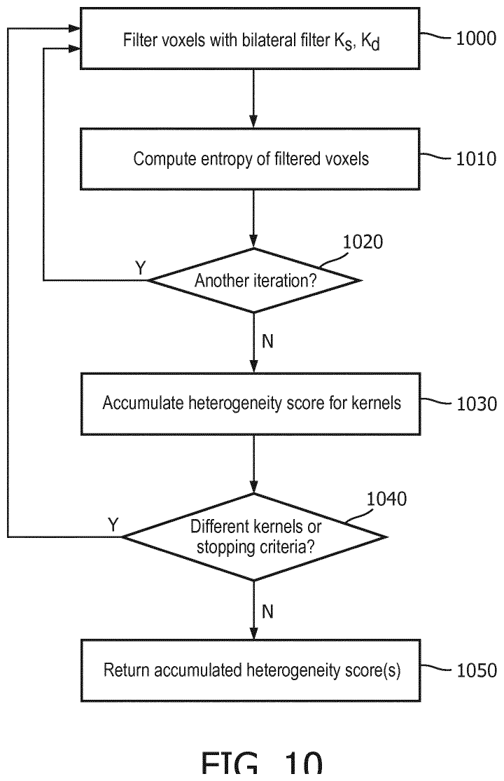

With reference to FIG. 9, an embodiment of a method for quantifying spiculation of lesions in medical images is flowcharted. At 900, the configured processor 162 generates the patches 210 equidistant along the segmented boundary 212 according to the received parameters of the patch dimensions 214 and a center line or plane of each patch tangential to the segmented boundary 212. The distance between the patches 210 can be a function of the patch dimensions 214, that is a function of y, such as y/2. The patches 210 can be 2D or 3D. The patches 210 are rectilinear in shape, such as a rectangle in 2D, cuboid in 3D, and the like.

At 910, the configured processor 162 samples voxel values in a line in the corresponding patch parallel to the tangent of the segmented boundary 212. That is, for each line in the corresponding patch, the voxel values are sampled, or voxels in a plane in 3D. For example, the gray scale or HU values are sampled in a CT image for one line in one patch and the one line is parallel to the tangent line to the segmented boundary 212.

At 920, the configured processor 162 fits the line 350 to the sampled voxel values 340. The fitted line 350 can include a straight line or a low order polynomial. For example, the configured processor 162 using a least square regression fits a straight line to voxels sampled along the sampled line of the patch.

At 930, the configured processor 162 computes the result 142 of a spiculation score, which adds one to the spiculation score for each voxel value that exceeds a predetermined threshold T difference from the fitted line 350. That is, residual values from the fitted line, such as from a least squares regression, are compared to the threshold, and a count of the number of residual values exceeding the threshold determines the spiculation score for the line or plane of sampled voxel values.

At 940, the configured processor 162 repeats act 910-930 for each line or plane in the patch 210. The acts 910-930 for each line or plane in the patch 210 can be performed in parallel.

At 950, the configured processor 162 repeats act 910-940 for each patch 210. The acts 910-940 for each patch 210 can be performed in parallel.

At 960, the configured processor 162 accumulates the spiculation score for the patch dimensions 214 and threshold T. That is, the spiculation score is the sum of the spiculation score for each sampled line of each patch. In some embodiments, the spiculation score is alternatively represented as a set of feature vectors at a level of granularity of the sampled line or the patch level.

At 970, the configured processor 162 repeats act 910-960 for each set of patch dimensions 214 and threshold T. The acts 910-960 for computing a spiculation score can be performed in parallel.

At 980, the configured processor 162 returns the spiculation score for each set of patch dimensions 214 and threshold T. The spiculation score can be returned as a single value or a set of vectors.

With reference to FIG. 10, an embodiment of a method for quantifying heterogeneity of lesions in medical images is flowcharted. At 1000, the configured processor 162 filters voxels within the segmented lesion with a bilateral filter. In some embodiments, the bilateral filter is extended to a multilateral filter, such as with spectral images and each additional kernel corresponds to a spectral channel, i.e. different energy spectrum.

At 1010, the configured processor 162 computes the entropy for the filtered voxels.

At 1020, the configured processor 162 iteratively repeats acts 1000-1010 according to a stopping criteria. The stopping criteria can include a fixed number of iterations or a threshold difference between successive entropy values below which the acts are not iteratively performed. The number of iterations or threshold can be received as parameters.

At 1030, the configured processor 162 accumulates the heterogeneity score according to the computed values. The heterogeneity score can include a single result, such as an area under a curve fitted to the computed entropy scores as a function of the iteration. The heterogeneity score can include a set of vectors, which represent the entropy score according to the iteration.

At 1040, the configured processor 162 can perform acts 1000-1030 for different kernels or stopping criteria. The acts 1000-1030 for each of the different kernels and/or stopping criteria can be performed in parallel.

At 1050, the configured processor 162 returns the heterogeneity scores. The heterogeneity score can include a single result or a set of vectors.

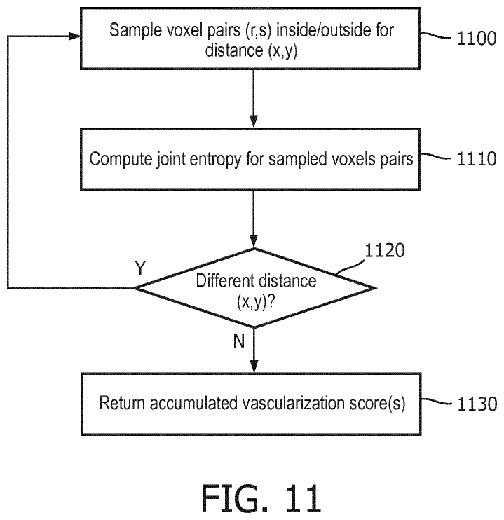

FIG. 11 flowcharts an embodiment of a method for quantifying vascularization of lesions in medical images. At 1100, the configured processor 162 samples voxel pairs (r, s) inside and outside the segmented contour 600 of the lesion 102. The voxel pairs (r, s) are selected according to an inside distance 602 x and an outside distance y 604 from the segmented contour 600. In some embodiments, x and y are the same. The voxel pairs (r, s) are selected along a line orthogonal to the segmented contour 600.

At 1110, the configured processor 162 computes the joint entropy 704 for the voxel pairs (r, s).

At 1120, the configured processor 162 can perform acts 1110-1120 for different distances x, y. The acts 1000-1030 for each of the different distances can be performed in parallel.

At 1130, the configured processor 162 returns a vascularization score. The vascularization score can include a single result, such as an area under a histogram of the computed joint entropy histograms 702, 712, 722, or a set of vectors that represent the individual joint entropies.

The above may be implemented by way of computer readable instructions, encoded or embedded on a computer readable storage medium, which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention is constructed as including all such modifications and alterations insofar as they come within the scope of the appended claims or the equivalents thereof. The word “comprising” does not exclude other elements or steps, and the indefinite article “a” or “an” does not exclude a plurality.

1. A system, comprising:
a segmenter configured to segment a lesion in a medical image; and
a quantification tool configured to quantify an aspect of the segmented lesion according to a set of parameters, wherein the quantified aspect includes at least one of spiculation, heterogeneity, and vascularization.

2. The system according to claim 1, wherein the quantified aspect is selected based on at least one of a protocol, a user preference, and a manual selection.

3. The system according to claim 1, wherein the quantification tool includes a spiculation quantifier configured to:
generate rectilinear patches spaced equidistant along and orthogonal to a segmented contour of the segmented lesion, wherein a centerline of the patches is tangential to the segmented contour; and
compute a spiculation score according to a number of voxels sampled along each line within each patch parallel to a tangent of the segmented contour which are a predetermined threshold difference from a corresponding fitted line.

4. The system according to claim 3, wherein the spiculation quantifier is further configured to return a result which includes at least one of:
a spiculation score that comprises a sum of spiculation scores for each line of each patch;
a spiculation score that comprises a set of vectors with dimensions of the patches;
a spiculation score that comprises a set of vectors with dimensions of the patches, and an individual patch spiculation score; and
a spiculation score that comprises a set of vectors with dimensions of the patches, and an individual line spiculation score of an individual patch.

5. The system according to claim 1, wherein the quantification tool includes a heterogeneity quantifier configured to:
iteratively filter voxels within the segmented lesion using at least one of a bilateral filter and a multilateral filter; and
compute an entropy for the filtered voxels with each iteration.

6. The system according to claim 5, wherein the heterogeneity quantifier is further configured to return a result which includes at least one of:
a heterogeneity score that comprises an area defined by a curve of the computed entropies of the filtered voxels according to the iteration; and
a heterogeneity score that comprises a set of vectors with the computed entropy of the filtered voxels and the iteration.

7. The system according to claim 1, wherein the quantification tool includes a vascularization quantifier configured to:
sample voxel pairs at an inside distance and an outside distance from a segmented contour, wherein the sampled voxel pairs are along a line orthogonal to the segmented contour; and
compute a joint entropy from the sampled voxel pairs.

8. The system according to claim 7, wherein the vascularization quantifier is further configured to return a result which includes at least one of:
a vascularization score comprising an area under a histogram of the computed joint entropy at different distances; and
a vascularization score comprising a set of vectors with the computed joint entropy of the distances.

9. The system according to claim 1, wherein the medical image is generated by a medical imaging device comprising at least one modality selected from computed tomography, magnetic resonance, and ultrasound.

10. A method, comprising:
segmenting a lesion in a medical image; and
quantifying an aspect of the segmented lesion according to a set of parameters, wherein the quantified aspect includes at least one of spiculation, heterogeneity, and vascularization.

11. The method according to claim 10, wherein the quantified aspect is selected by at least one of a protocol, a user preference, and a manual selection.

12. The method according to claim 10, wherein quantifying comprises:
generating rectilinear patches spaced equidistant along and orthogonal to a segmented contour of the segmented lesion, wherein a centerline of the patches is tangential to the segmented contour; and
computing a spiculation score according to a number of voxels sampled along each line within each patch parallel to a tangent of the segmented contour which are a predetermined threshold difference from a corresponding fitted line.

13. The method according to claim 12, further including:
returning a result which includes at least one of:
a spiculation score that comprises a sum of spiculation scores for each line of each patch;
a spiculation score that comprises a set of vectors with dimensions of the patches;
a spiculation score that comprises a set of vectors with dimensions of the patches, and an individual patch spiculation score; and
a spiculation score that comprises a set of vectors with dimensions of the patches, and an individual line spiculation score of an individual patch.

14. The method according to claim 10, wherein quantifying comprises:
iteratively filtering voxels within the segmented lesion using at least one of a bilateral filter and a multilateral filter; and
computing an entropy for the filtered voxels with each iteration.

15. The method according to claim 10, further including:
returning a result which includes at least one of:
a heterogeneity score that comprises an area defined by a line of the computed entropies of the filtered voxels according to the iteration; and
a heterogeneity score that comprises a set of vectors with the computed entropy of the filtered voxels and the iteration.

16. The method according to claim 10, wherein quantifying comprises:
sampling voxel pairs at an inside distance and an outside distance from a segmented contour, wherein the sampled voxel pairs are along a line orthogonal to the segmented contour; and
computing a joint entropy from the sampled voxel pairs.

17. The method according to claim 16, further comprising:
returning a result which includes at least one of:
a vascularization score comprising an area under a histogram of the computed joint entropy at different distances; and
a vascularization score comprising a set of vectors with the computed joint entropy of the distances.

18. A non-transitory computer-readable storage medium having one or more executable instructions stored thereon which, when executed by one or more processors, cause the one or more processors to:
segment a lesion in a medical image; and
quantify an aspect of the segmented lesion according to a set of parameters, wherein the quantified aspect includes at least one of spiculation, heterogeneity and vascularization.

19-22. (canceled)
